(19) United States
(12) Patent Application Publication
(10) Pub. No.: US 2004/0028760 A1
(43) Pub. Date: Feb. 12, 2004

(54) Method of treating inflammation and inflammation-related pain

(75) Inventors: Muraleedharan Nair, Okemos, MI (US); Rafikali Momin, Guelph (CA)

Correspondence Address: MCLEOD & MOYNE, P.C., 2190 COMMONS PARKWAY, OKEMOS, MI, 48864, US

(73) Assignee: Michigan State University

(21) Appl. No.: 10/435,930

(22) Filed: May 12, 2003

Related U.S. Application Data
(63) Continuation of application No. 10/077,903, filed on Feb. 18, 2002, now abandoned.

(51) Int. Cl. A61K 35/78

(52) U.S. Cl. 424/776

(57) ABSTRACT

A method for treating cycloxygenase enzyme inflammation and inflammation pain is disclosed. In one embodiment, this method comprises the step of treating an inflammation patient with a specific amount of carrot seed or carrot seed extract, wherein inflammation is reduced and pain is decreased.

### CROSS-REFERENCE TO RELATED APPLICATION

### STATEMENT REGARDING FEDERALLY SPONSORED RESEARCH OR DEVELOPMENT

### BACKGROUND OF THE INVENTION

Carrot (D. carota L.) is an annual or biennial herb cultivated throughout the temperate regions of the world. Carrots are one of the important food crops in the world, and are used extensively for canned, frozen and dehydrated products. Even though carrot is widely used as a vegetable, other parts of this plant are used in traditional medicine for the treatment of a broad spectrum of ailments. Carrot seeds, commonly known as carrot fruits, are well known for its use as carminative, diuretic, stimulant, and in the treatment of digestive disorder (Volak, et al., 1984). The essential oil from the seeds was also studied for its hypotensive, cardiac, anticonvulsant and anti-fertility activities (Kamboj, 1988; Dhar, 1990; Chopra, et al., 1958; Halim, et al., 1988). Seed oils of some selected varieties were reported as an antibacterial (Syed, et al., 1986) and fungicidal (Guerin and Reveillere, 1985; Dwivedi, et al., 1991) agents. Carrot seed oil is widely used as flavoring agent in food products, grape wine, nonalcoholic beverages (Bodrug, 1982) and in perfumery (Guenther, 1950).

The conversion of arachidonic acid to prostaglandins, catalyzed by cyclooxygenase enzymes COX-I and COX-II, is well documented (O'Banion, 1999). The COX-I enzyme is constitutively expressed in many tissues. COX-II enzyme is normally not expressed in most tissues but observed mainly in inflamed tissues. Inhibition of COX-I enzyme, which reduces the production of prostaglandins in the stomach, can cause gastric ulceration. However, the selective inhibition of COX-II enzyme can cause minimal side effects. Therefore, researchers are investigating numerous natural products for selective inhibitors to COX-II enzymes.

Carrot seeds have been studied for their chemical composition. However, limited research has been done to establish any link between its constituents and phytoceutical properties. The present invention discloses that extracts of carrot seeds yield compounds that are selective to COX-II enzyme inhibition and useful for the reduction of inflammation.

### BRIEF SUMMARY OF THE INVENTION

In one embodiment, the present invention is a method of treating cyclooxygenase enzyme mediated inflammation and inflammation-related pain. The method comprises the step of treating an inflammation patient with a sufficient amount of carrot seed or carrot seed extract wherein inflammation is reduced and inflammation-related pain is decreased.

In a preferred form of the present invention, the patient is treated with either carrot seed, carrot seed extract or powdered or ground carrot seed.

It is a feature of the present invention that one may treat inflammation and inflammation-related pain with a naturally occurring product.

Other features, objects and advantages of the present invention will become apparent after one of skill in the art has reviewed the specification, claims and drawings.

### BRIEF DESCRIPTION OF THE SEVERAL VIEWS OF THE DRAWINGS

FIG. 1 illustrates bioactive compounds from D. carota seeds and 2,4,5-trimethoxybenzoicacid (5).

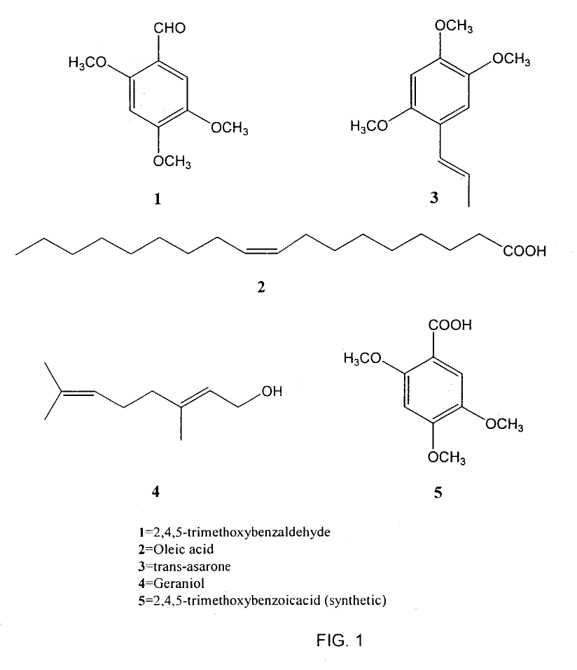

FIG. 2 is a bar graph showing in vitro COX-I and COX-II inhibitory activities of compounds 1-4 and 2,4,5-trimethoxybenzbic acid (5) at 100 μg mL⁻¹ and Ibuprofen, Naproxen, Asprin, Celebrex and Vioxx at 2.06, 2.52, 180, 1.67 and 1.67 μg mL⁻¹, respectively. Vertical bars represent the standard deviation of each data point (n=2).

### DETAILED DESCRIPTION OF THE INVENTION

The present invention is a method of treating cyclooxygenase enzyme, preferably COX-II enzyme or COX-I mediated inflammation or inflammation pain. In a preferred form of the present invention, the method treats COX II enzyme mediated inflammation or inflammation pain. In one embodiment, the invention comprises the step of providing a patient with a sufficient amount of carrot seed or carrot seed extract, wherein the patient's inflammation or pain is reduced. In a particularly advantageous form of the invention, the patient is afflicted with arthritis or gout related pain.

In the present invention, one would first identify a patient with inflammation and inflammation-related pain. In addition to arthritis or gout, other examples of inflammation-related pain would be exercise, injury, surgery, headaches, and menstrual pain.

In one embodiment, the patient would be supplied with carrot seeds, preferably in a dose of 10-400 grams per day, preferably 20-200 grams per day. Applicants envision that the carrot seeds do not necessarily need to be intact for successful treatment. For example, the carrot seeds could be ground, powdered, pulped, or liquefied and may be pressed into pill or in capsule form. (When applicants refer to “carrot seeds that are not intact,” applicants mean to include any version of non-whole carrot seeds.) The carrot seed material may be combined with pharmaceutically acceptable carriers or other compositions suitable to make successful pills, capsules, or other oral medications.

Preferably, one would administer the dose as follows: One would orally take 10-50 mg of active ingredients (preferably 20-40 mg) twice a day. By “active ingredients” Applicants mean any combination of compounds.

Additionally, Applicants envision that the carrot seed could be extracted to create a composition with an enhanced amount of anti-inflammatory compounds 1-3. Applicants have described a particular extraction below in the Examples. The extract described below is useful for isolating the various anti-inflammatory compounds 1-3. However, Applicants believe that one would not necessarily need to separate these compounds to have a successful therapeutic composition.

In one embodiment of the present invention, one would combine the carrot seed extract with carrot seeds, either intact or non-intact. Most preferably, the extract is combined with pulped carrot seed.

For example, one could crudely extract the carrot seeds in the following manner: Powdered carrot seeds may be extracted with hexane. The hexane is then removed. Preferred, one would fractionate the crude extract to remove some or most of the triglycerides, such as by use of a silica column or other chromatographic methods.

A “carrot seed extract” of the present invention is an extract taken from carrot seed that will preferably contain concentrated versions of the anti-inflammatory compounds 1-3 described below. The compounds may not be at the same ratio as are normally found in carrot seed and may not all be present. Preferably, 1 kg carrot seed would yield 125 grams of extract free from hexane. If triglycerides are removed, the total weight of active ingredients may be between 3-5 grams.

In another form of the invention, one would treat the patient with COX-II enzyme-mediated inflammatory pain with a mixture of compounds selected from the group consisting of 2,4,5-trimethoxybenzaldehyde, oleic acid, and trans-asarone. In a particularly adventageous form of the invention, one would treat the patient with 2,4,5-trimethoxybenzaldehyde.

In another form of the present invention, one would combine the carrot seeds, powdered carrot seeds or carrot seed extract with a second pain relieving compound, such as the compositions described below.

### EXAMPLES

1. In general

The examples below disclose that cyclooxygenase enzymes inhibitory assay directed investigation of Daucus carota seed extracts resulted in the isolation and characterization of compounds, 2,4,5-trimethoxybenzaldehyde (1), oleic acid (2), trans-asarone (3) and geraniol (4). Compounds 1-4 showed 3.32, 45.32, 46.15, and 3.15% of prostaglandin H endoperoxide synthase-I (COX-I) inhibitory activity and 52.69,68.41,64.39 and 0% prostaglandin H endoperoxide synthase-II (COX-II) inhibitory activity, respectively at 100 μg mL⁻¹. Compound 1 showed selectivity towards COX-II enzyme inhibition at 100 μg mL⁻¹. The COX-II/COX-I ratio for compound 1 was 17.68 at 100 μg mL⁻¹ compared to solvent control.

Ibuprofen, Naproxen, Aspirin, Celebrex and Vioxx at concentrations of 2.06, 2.52, 180, 1.67 and 1.67 μg mL⁻¹, respectively, gave COX-II/COX-I ratios of 1.13, 0.92, 0.24, 16 and 75%, respectively. The inhibition of COX-I and COX-II enzymes by compounds 1-3 at 100 μg mL⁻¹ were comparable to Ibuprofen, Naproxen, Celebrex and Vioxx assayed at 2.52, 2.06 and 180 μg mL⁻¹ concentrations, whereas Celebrex and Vioxx were tested at 1.67 μg mL⁻¹, respectively.

2. Materials and Methods

Plant Material

Carrot seeds were provided by Asgrow Seed Company, Kalamazoo, Mich. and stored at −20° C. until extraction.

General Experimental Procedures

¹H- and ¹³C NMR, spectra were recorded on Varian INOVA 300 or 500 MHz spectrometers. ¹³C NMR spectra were recorded at 75 or 126 MHz. Chemical shifts were recorded in CDCl₃, and the values are reported in δ (ppm) based on δ residual of 7.24 for ¹H NMR and 77 for ¹³C NMR. Coupling constants J, are in Hz. The silica gel used for MPLC was Merck Silica gel 60 (30-70 μm particle size). TLC plates (Analtech, Silica gel GF, coated on PE sheets, 200 microns), and preparative TLC plates (Analtech, Silica gel, 20×20 cm², 250, 500 and 1000 micron) after developing, were viewed under UV light (254 and 366 nm). All organic solvents used were ACS reagent grade (Aldrich Chemical Co., Inc., Milwaukee, Wis.). Authentic samples of 2,4,5-trimethoxybenzaldehyde; 2,4,5-trimethoxybenzoicacid and trans-asarone as well as Ibuprofen and Naproxen were purchased from Sigma-Aldrich Chemical Co., Inc. St. Louis, Mo.). Celebrex® capsules and Vioxx® tablets were physician's professional samples provided by Dr. Subhash Gupta, Sparrow Pain center, MI.

Extraction and Isolation

The ground carrot seeds (1 kg) were sequentially extracted with hexane, EtOAc, and MeOH (1.5×4, 24 h each) and yielded 121.1, 57.8 and 25.1 g of residue, respectively, after evaporating the solvents. An aliquot of the hexane extract (32 g) was stirred with MeOH and filtered to yield MeOH soluble (4.84 g) and insoluble (26 g) fractions. The bioactive MeOH soluble fraction (3.09 g) was further fractionated by MPLC on silica gel (Sanki Engineering Ltd., Model LBP-V pump operating at 1-15 psi., Chemco MPLC tayperling type glass column, 35×4 cm²) using hexane with increasing amount of acetone and finally with MeOH as eluting solvents. Fractions collected were A eluted with 100% hexane and hexane:acetone (8:1, 720 mL, 220 mg), B (155 mL, 1843 mg) and C (75 mL, 145 mg) with hexane:acetone (8:1), D (75 mL, 74 mg), E (75 mL, 15 mg) and F (75 mL, 17 mg) with hexane:acetone (4:1), G (195 mL, 165 mg) with hexane:acetone (4:1 and 1:1) and H (165 mL, 300 mg) with 100% acetone and MeOH.

Compound 1 (7.8 mg) was yielded from the purification of fraction H (40 mg) by preparative TLC using hexane:acetone (4:1×3) as the mobile phase. Similarly, compound 2 was purified from fraction G by preparative TLC (hexane: EtOAc, 3:1). Fraction B was further subjected to MPLC on silica gel using hexane with increasing amount of acetone and yielded six fractions (I-VI). The fraction IV (73 mg), eluted with hexane:acetone (15:1), was further purified by preparative TLC (hexane:chloroform:toluene:MeOH, 3:2:2:0.1) and afforded pure compound 3 (5.9 mg). Compound 4 (20.3 mg) was isolated from fraction A (40 mg) by preparative TLC (100% hexane).

Compound 1: ¹H NMR (CDCl₃, 300 MHz)δ 3.86 (s, 3H, 4-OCH₃), 3.91 (s, 3H, 5-OCH₃), 3.96 (s, 3H, 2-OCH₃), 6.47 (s, 1H, H-3), 7.31 (s, 1H, H-6), 10.3 (s, 1H, —CHO).

Compound 2: ¹H NMR (CDCl₃, 300 MHz) δ 0.82 (t, 3H, H-18), 1.20-1.30 (m, 20H, H(4-7, 12-17)), 1.61 (m, 2H, H-3), 2.03 (m, 4H, H-8,11), 2.34 (m, 2H, H-2), 5.35 (m, 2H, H-9, 10).

Compound 3: ¹H NMR (CDCl₃, 300 MHz) δ 1.88 (dd, 3H, J=7.0, 2.0 Hz, H-9), 3.80 (s, 3H, 4-OCH₃), 3.85 (s, 3H, 5-OCH₃), 3.87 (s, 3H, 2-OCH₃), H-8), 6.47 (s, 1H, H-3), 6.64 (dd, 1H, J=16.0, 2.0 Hz, H-7), 6.95 (s, 1H, H-6); ¹³C NMR (CDCl₃, 75 MHz) δ 18.81 (C-9), 56.10 (2-OCH₃), 56.46 (4-OCH₃), 56.73 (5-OCH_{3),} 97.88 (C-3), 118.94 (C-1), 143.51 (C-5), 149.60 (C-4), 150.35 (C-2).

Compound 4: ¹H NMR (CDCl_{3, 300} MHz) δ 1.62 (s, 3H, H-9), 1.68 (s, 3H, H-10), 1.70 (s, 3H, H-8), 2.1 (m, 4H, H-4, 5), 4.7 (m, 2H, H-1), 5.10 (t, 1H, J=6 Hz, H-6), 5.40 (m, 1H, H-2).

Cyclooxygenase Inhibitory Assay

Purified compounds 1-4 from carrot seeds and authentic samples of 2,4,5-trimethoxybenzaldehyde; 2,4,5-trimethoxybenzoic acid (5) and trans-asarone were assessed for their cyclooxygenase inhibitory activity at 37° C. by monitoring the initial rate of O₂ uptake using an O₂ electrode (Instech Laboratories, Inc., 5209 Militia Road, Plymouth Meeting PA 19462-1216) (Smith, et al., 2000; Laneuville, et al., 1994). Prostaglandin H synthase isozymes-I (PGHS-I or COX-I) was prepared from ram seminal vesicles. PGHS-II or COX-II enzyme for the assay was prepared from lysates of cloned insect cell with human PGHS-II enzyme. Each assay mixture contained 600 μL of 0.1 M Tris buffer (pH 7), 1 mM phenol, 17 μg hemoglobin and 10 μL of COX-I or 20-30 μL of COX-II enzymes. Crude extracts or pure compounds were preincubated with 25-100 μg mL⁻¹ in DMSO for 5-minutes with COX-I or COX-II enzymes in the assay chamber at 37° C. Cyclooxygenase inhibitory activities were initiated by the addition of 1.64 μM arachidonic acid to the tst compound-enzyme mixture at 37° C. Instantaneous inhibition of the enzyme was determined by monitoring the initial rate of O₂ uptake using an O₂ electrode. Ibuprofen, Naproxen and Aspirin were assayed at their IC₅₀ values, 2.52, 2.06 and 180 μg ML⁻¹ concentrations, respectively, whereas Celebrex and Vioxx were tested at 1.67 μg mL⁻¹.

3. Results

The ¹H-NMR of compound 1 gave a singlet at 10.3 ppm, which was not exchangeable with D₂O suggested that it contained an aldehydic proton in the molecule. Three singlets at 3.86, 3.91 and 3.96 ppm, integrated for three protons each, indicated the presence of three methoxy groups. In addition, two singlets at 6.47 and 7.31 ppm suggested the presence of a tetra substituted aromatic ring in the molecule. The ¹H NMR spectrum of 1 was identical to the published spectra of 2,4,5-trimethoxybenzaldehyde (Nowamaki and Kuroyanagi, 1996).

¹H NMR spectral data were sufficient to determine the structure of compound 2. A multiplet at 5.35 ppm, integrated for two protons suggested the presence of a double bond in the molecule. Protons resonated between δ 2.34 and 1.61 were assigned to methylene protons of α- and β- to a carboxylic group. The —CH₂ protons observed between δ 1.20 and 1.30 in the ¹H-NMR of 2 were assigned to protons of C4-C8 and C12-C17. A triplet, integrated for three protons at δ 0.83, was assigned to a CH₃ group in 2. Based on the ¹H NMR spectral data, compound 2 was identified as oleic acid (Arnhold, et al., 1995).

Compound 3 was identified as trans-asarone by analyses of its ¹H and ¹³C NMR spectral data and by comparison to the published spectral values (Siergiejczyk, et al., 2000) for trans-asarone. Similarly, the structure of compound 4 was confirmed as geraniol by ¹H NMR spectral data and by comparison of the published spectral data (De Haan and Van de Ven, 1971; Bunton, et al., 1972).

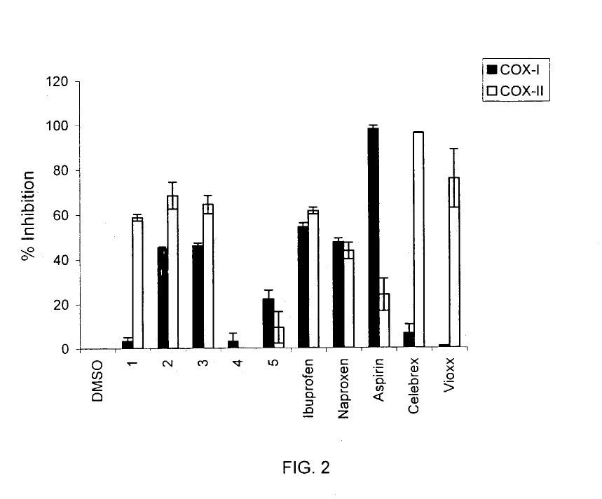

Compounds 1-4 were evaluated for their cyclooxygenase inhibitory activities using COX-I and -II enzymes at 25-100 μg mL⁻¹. Compounds 1-3 demonstrated 3.32, 45.32 and 46.15% of COX-I inhibition (FIG. 2) and 52.69, 68.41 and 64.39% of COX-II inhibition (FIG. 2), respectively, when assayed at 100 μg mL⁻¹. Compound 1 showed selective inhibition of COX-II enzyme. The COX-II:COX-I ratio for compound 1 was 17.68 at the concentration of 100 μg mL⁻¹ compared to 1.13, 0.92, 0.24, 16 and 75 for Ibuprofen, Naproxen, Aspirin, Celebrex and Vioxx at concentrations of 2.06, 2.52, 180, 1.67 and 1.67 μg mL⁻¹, respectively. Compound 1 was further evaluated for COX-II activity at lower concentrations and observed >30% inhibition of COX-II enzyme at 25 μg mL⁻¹. Compounds 2 and 3, at 100 μg mL⁻¹, showed similar COX-I and -II inhibitory activities to the commercial anti-inflammatory drugs. The COX-II/COX-I ratio for compounds 2 and 3 at 100 μg mL⁻¹ were 1.51 and 1.4, which is comparable to the ratio for Ibuprofen and Naproxen when tested at 2.06, 2.52 ppm. Compound 4 was not active when tested at 100 μg mL⁻¹ in both COX enzymes inhibitory assays.

The commercially available and synthetic compounds, 2,4,5-trimethoxybenzaldehyde; 2,4,5-trimethoxybenzoicacid (5) and trans-asarone purchased from Sigma-Aldrich Chemical Co., Inc. were also tested for COX-I and COX-II inhibitory activities at 25-100 μg mL⁻¹ concentrations. Synthetic 2,4,5-trimethoxybenzaldehyde showed slightly lower COX-II activity than natural form (1) isolated from carrot seeds, whereas COX-I and COX-II inhibition of compound 3 and synthetic trans-asarone were similar to natural products isolated from carrot seeds. The lower activity of the synthetic compound 1 was accounted to the presence of small amount of corresponding acid due to oxidation under storage. 2,4,5-trimethoxybenzoicacid didn't exhibit any inhibition of COX-I or COX-II enzymes at 100 μg mL⁻¹ concentration.

4. Discussion

Compounds 2 and 3 isolated from carrot seeds showed comparable COX inhibition to some of the over the counter (OTC) anti-inflammatory drugs. Compound 1 exhibited selective inhibition of COX-II enzyme. The COX-II/COX-I ratio for compound 1 was 17.68 at a test concentration of 100 μg mL⁻¹ compared to the solvent control. This value is better than the COX-II/COX-I ratios for Ibuprofen, Naproxen, Aspirin and Celebrex at their respective test concentrations. Compound 4 did not inhibit COX-I or -II enzymes at 100 μg mL⁻¹ concentration. Among the authentic samples tested, only 2,4,5-trimethoxybenzaldehyde and trans-asarone exhibited COX enzyme inhibitory activities. The high COX-II/COX-I ratio of compound 1, moderate COX enzymes inhibitory activity of 3 and the lack of activity of 2,4,5-trimethoxybenzoicacid (5) suggested that the methoxy groups are not a major contributing factor for activity. Greca, et al. (1992) studied the structure-activity relationship of phenylpropanoids as growth inhibitors of green alga Selenastrum capricornutum. They concluded that the activity of phenylpropanoids depends on the number and positions of methoxy groups in the ring. They also observed that the activity increased from the monomethoxy to the trimethoxy derivatives and methoxy groups at ortho and para to the side chain provided the strongest biological activity. The observed cyclooxygenase enzyme inhibitory activities of Compounds 1 and 3 might be attributable to both aldehydic and propenyl functionalities, rather than just methoxy groups in these molecules. Compound 2, oleic acid, gave 45.32 and 68.41% of COX-I and COX-II inhibitory activities, respectively. Ringbom, et al. (2001) studied COX-I and COX-II inhibitory effects of some naturally occurring fatty acids. However, they found oleic acid was not COX-I or COX-II inhibitory at less than 500 μM concentrations contrary to our finding.

This is the first report of the cyclooxygenase enzyme inhibitory activities of Compounds 1 and 3. The selective COX-II inhibitory activity of Compound 1 is significant and suggests that carrot seeds could be consumed for the prevention of arthritic or gout related pains and suggest that the seeds would alleviate other COX-II enzyme mediated inflammatory pain.

### REFERENCES

Arnhold, J., Panasenko, O. M., Schiller, J., Vladimirov, Yu. A., Arnold, K., The action of hypochiorous acid on phosphatidylcholine liposomes in dependence on the content of double bonds. Stoichiometry and NMR analysis. Chem. Phys. Lipids. 78:55-64, 1995.

Bodrug, M. V., Status and prospects of study and use of aromatic plants in the Moldavian-SSR, USSR. Rastit. Resur. 18:558-560,1982.

Bunton, C. A., Hachey, D. L., Leresche, J. P., Deamination of Nerylamine and Geranylamine. J. Org. Chem. 37:4036-4039,1972.

Chopra, R. N., Chopra, I. C., Handa, K. L., Kapur, L. D., Chopra's Indigeneous Drugs of India, 2^{nd} Edn. U. N. Dhar and Sons Pvt. Ltd., Calcutta, India. pp. 504,1958.

De Haan, J. W., Van de Ven, L. J. M., Z-E Conformational Isomerism of Nerol, Geraniol and their Acetates. Tetrahedron Lett. 29:2703-2706, 1971.

Dhar, V. J., Studies on Daucus carota seeds. Fitoterapia. 61:255-258, 1990.

Dwivedi, S. K., Pandey, V. N., Dubey, N. K., Effect of essential oils of some higher plants on Aspergillus flavus Link. Infesting stored seeds of Guar cyamopsis-tetragonoloba L. Flavour Fragrance J. 6:295-298, 1991.

Greca, M. D., Monaco, P., Pollio, A., Previtera, L., Structure-activity relationship of phenylpropanoids as growth inhibitors of the green alga Selenastrum capricornutum. Phytochemistry. 31:4119-4123,1992.

Guenther, E., The Essential Oils. Vol. IV, D. Van Nostrand Company, New York, USA. pp. 585, 1950.

Guerin, J. C. and Reveillere, H. P., Antifungal activity of plant extracts used in therapy. Part 2: Study of 40 plant extracts against 9 fungi species. Ann. Pharm Fr. 43:77-81,1985.

Halim, A. F., Mashaly, M. M., Sandra, P., Analysis of the fruit essential oil of Daucus carota L. var. Boissieri Schweinf. 1^{st} Anglo-Egyptian Conference of Pharm. Sci., Alexandrin, Egypt, November 15-17,1988.

Kamboj, V. P., A review of Indian medicinal plant with interceptive activity. Indian J. Med. Res. 87:336-355,1988.

Nawamaki, K. and Kuroyanagi, M., Sesquiterpenoids from Acorus calamus as germination inhibitors. Phytochemistry. 43:1175-1182,1996.

O'Banion, M. K., Cyclooxygenase-2: molecular biology, pharmacology, and neurobiology. Crit. Rev. Neurobiol. 13:45-82, 1999.

Siergiejczyk, L., Paplawski, J., Lozowicka, B., Dubis, A., Lachowska, B., ¹H and ¹³C NMR spectral analysis of (E)-asarone and its isomers. Magn. Reson. Chem. 38:1037-1038, 2000.

Smith, T., Leipprandt, J., De Witt, D., Purification and Characterization of the Human Recombinant Histidine-Tagged Prostaglandin Endoperoxide H Synthase-1 and -2. Arch. Biochem. Biophysics. 375:195-200, 2000.

Syed, M., Sabir, A. W., Chaudhary, F. M., Bhatty, M. K., Antimicrobial activity of the essential oils of Umbelliferae Part II. Trachyspermum amni, Daucus carota, Anethum graveolens and Apium graveolens oils. Pak. J. Sci. Ind. Res. 29:189-192,1986.

Volak, J., Stodola, J., Severa, F., Plantes Medicinales. Grund-Paris, pp. 140, 1984.

We claim:

1. A method of treating cyclooxygenase enzyme-mediated inflammation, comprising the step of treating an inflammation patient with a sufficient amount of carrot seed or carrot seed extract, wherein inflammation is reduced.

2. A method of treating COX I or COX-II enzyme mediated inflammation, comprising the step of treating an inflammation patient with a sufficient amount of carrot seed or carrot seed extract, wherein inflammation is reduced.

3. The method of claim 2, wherein the inflammation is mediated by COX-II.

4. The method of claim 1, wherein the patient suffers from pain related to a disease selected from arthritis or gout.

5. The method of claim 1, wherein the patient is treated with carrot seeds at a dosage of 10-50 mg active ingredients.

6. The method of claim 1 wherein the patient is treated with intact carrot seeds.

7. The method of claim 1 wherein the patient is treated with carrot seeds that are not intact.

8. The method of claim 7 wherein the extract is combined with intact or non-intact carrot seeds.

9. The method of claim 8 wherein the extract is combined with pulped carrot seeds.

10. The method of claim 1, wherein the patient is treated with carrot seed extract.

11. The method of claim 10, wherein the extract is created by hexane extraction.

12. The method of claim 1 additionally comprising the step of combining the carrot seed or carrot seed extract with a second pain relieving compound.

13. A method of treating inflammation, comprising:
the step of treating an inflammation patient with a compound selected from the group consisting of 2,4,5-trimethoxybenzaldehyde, oleic acid, and trans-asarone, wherein inflammation is reduced.

14. The method of claim 13, wherein the treatment is at a dosage of 10-50 mg per dose.

15. The method of claim 13, wherein treatment is with 2,4,5-trimethoxybenzaldehyde.

16. A composition useful for the treatment of inflammation or pain wherein the composition comprises carrot seed extract.

17. The composition of claim 16 wherein the active ingredients are at 10-50 mg per dose.

18. The composition of claim 16 additionally comprising a second pain-relieving compound.
